(19) United States
(12) Patent Application Publication
(10) Pub. No.: US 2025/0082387 A1
(43) Pub. Date: Mar. 13, 2025

(54) DIGITALLY CONTROLLED APPLICATION OF A CRYOGENIC LIQUID DROP BY DROP

(71) Applicant: WUERTH ELEKTRONIK GMBH & CO. KG, Niedernhall (DE)

(72) Inventor: Andreas GIMMER, Rickenbach (DE)

(73) Assignee: WUERTH ELEKTRONIK GMBH & CO. KG, Niedernhall (DE)

(21) Appl. No.: 18/247,560

(22) PCT Filed: Sep. 8, 2021
(86) PCT No.: PCT/EP2021/074628

§ 371 Date: Mar. 31, 2023

(30) Foreign Application Priority Data
Sep. 8, 2020 (DE) 10 2020 123 415.5

(51) Int. Cl. A61B 18/02; A61B 18/00

(52) U.S. Cl. CPC A61B 18/0218; A61B 2018/0047; A61B 2018/00744; A61B 2018/00791

(57) ABSTRACT

The invention relates to a use of a cryogenic liquid. The use comprises a dropwise digitally controlled application of the cryogenic liquid to a surface for selectively influencing a temperature of the surface and/or an area located below the surface, in particular a layer or a substrate.

### DESCRIPTION

The invention relates to a use of a cryogenic liquid, an apparatus adapted to a corresponding use of a cryogenic liquid, and an apparatus for manufacturing a component comprising a corresponding device.

The application of cryogenic liquids to various surfaces for different purposes is known from the prior art. However, conventional methods often only allow rough control of the application process, which does not meet all of the necessary requirements.

In contrast, the present invention is based on the task of creating an improved use of a cryogenic liquid as well as an apparatus which is designed for a corresponding improved use of a cryogenic liquid.

The problem underlying the invention is solved in each case with the features of the independent patent claims. Embodiments of the invention are indicated in the dependent patent claims. Embodiments of the invention can be combined with each other provided that they are not mutually exclusive.

In one aspect, the invention relates to a use of a cryogenic liquid, characterized in that the cryogenic liquid is applied dropletwise to a surface in a digitally controlled manner for influencing, in particular for selectively influencing, a temperature of the surface.

According to embodiments, the use further comprises, for example, influencing, in particular selectively influencing, a temperature of a region underlying the surface, in particular a layer or a substrate.

Embodiments can have the advantage that they enable a targeted local thermo-kinetic influence on the surface to which the cryogenic liquid is applied or impinged droplet by droplet in a digitally controlled manner. For example, the application or the impingement of the cryogenic liquid droplets can serve a targeted, e.g., exclusively targeted, temperature influence of the applied surface, e.g., a surface of a substrate. By means of this targeted temperature influence, a temperature of the surface can be adjusted. For example, a two-dimensional spatial temperature gradient can be generated in a targeted manner. In addition, the application can serve, for example, a targeted temperature influence of an area below the applied surface, i.e., a three-dimensional area of an object whose surface is impinged with the cryogenic liquid. By means of this targeted temperature influence, a temperature of the area can be adjusted. For example, a three-dimensional spatial temperature gradient can be generated in a targeted manner. The term “application” or “impingement” is understood here to mean such a droplet-by-droplet digitally controlled application of the cryogenic liquid that the applied droplets of the cryogenic liquid influence the temperature of the “impinged” surface.

It is irrelevant whether the cryogenic liquid reaches the surface, remains on it or changes to a gaseous aggregate state during or immediately after the application process and leaves the surface again. In other words, the “application” or “impingement” may comprise an application of the droplets in which the applied droplets of the cryogenic liquid reach the “impinged” surface. For example, the droplets remain on the surface or change to a gaseous aggregate state, whereby the cryogenic liquid leaves the surface again. Thus, for example, a direct temperature influence of the surface by the cryogenic liquid can occur. Furthermore, the “application” or “impingement” may comprise an application of the droplets, in which the applied droplets of the cryogenic liquid change into the gaseous aggregate state during the application process. As a result of the transition to the gaseous aggregate state, the cryogenic liquid according to embodiments, for example, does not reach the “impinged” surface. The transition to the gaseous state of aggregation creates, for example, a temperature sink above the “impinged” surface, which extracts heat from the “impinged” surface. This heat extraction occurs, for example, via the atmosphere in which the surface is located. Thus, for example, an indirect temperature influence of the surface by the cryogenic liquid can take place.

Whether or not the droplets reach the surface may be determined, for example, by controlling or regulating one or more of the following parameters: Droplet size, exit velocity, distance of the printhead from the surface, angle of droplet delivery relative to the surface, ambient pressure, and/or ambient temperature.

Thus, for example, it can be specifically controlled whether, when and/or where the droplets reach the surface. This corresponds to a focusing of the cooling using the cryogenic liquid. If the cryogenic liquid reaches the surface, a stronger local cooling of the surface and/or a cooling extending deeper into the area below the surface can be achieved. If the cryogenic liquid does not reach the surface, a weaker cooling of the surface and/or a cooling extending less deeply into the area below the suface can be achieved locally. In addition, excessive stress on the surface due to direct contact of the surface with the cryogenic liquid can be avoided. Furthermore, undesirable, for example chemical interactions of the cryogenic liquid with the surface and/or with substances applied to the cooled surface can be avoided.

According to one embodiment, the temperature of the surface is measured during the application of the cryogenic liquid using a suitable, preferably non-contact, temperature measuring device. For example, an infrared thermometer can be used. The temperature can be measured once or preferably several times, in particular continuously during the application, e.g., to determine whether the temperature is within a desired range and, if not, to adjust one or more application parameters (e.g. distance, droplet number, droplet size, droplet emission velocity, droplet emission angle, etc.).

For example, a distribution scheme for distributing the cryogenic liquid on and/or over the surface may define where, when, how much cryogenic liquid to apply, and whether or not the applied cryogenic liquid should reach the surface. For example, the distribution scheme defines a spatial and/or temporal distribution of the cryogenic liquid. For example, the distribution scheme defines a spatially and/or temporally varying delivery amount of the cryogenic fluid. For example, the distribution scheme may define a distribution in which the delivery of the cryogenic fluid is modulated such that the cryogenic fluid reaches the surface at one or more locations while not reaching the surface at one or more other locations. Thus, for example, a distribution can be implemented which comprises both a direct and an indirect temperature influence on the surface in a locally targeted manner.

This thermo-kinetic influence on the surface can, for example, serve to prepare the impacted surface, i.e. a temperature setting, for a subsequent surface processing operation. For example, the thermo-kinetic influence on the surface can serve to influence the function of the impacted surface. Thus, the temperature setting can have an effect on physical, chemical and/or biological properties of the surface. For example, the thermo-kinetic influence on the surface can serve to influence the structure of the exposed surface.

Embodiments may have the advantage of applying a cryogenic liquid to the surface in a precise droplet-by-droplet manner. This enables fine-structured temperature control or cooling control on the corresponding surface. For example, the droplets may be droplets of small droplet size. For example, the droplets may be droplets of a droplet size of less than 100 picoliters (pl), preferably less than 10 pl. For example, it may be droplets of a droplet size of 1 pl to 10 pl, preferably 1 pl to 5 pl. A corresponding finely structured temperature control or temperature regulation on the surface makes it possible to control physical and/or chemical processes or reactions on the surface precisely or with pinpoint accuracy. Furthermore, by regulating the locally applied amount of cryogenic liquid, for example, the corresponding cooling effect can be controlled not only two-dimensionally but also three-dimensionally. For example, if more cryogenic liquid is applied locally, its cooling effect can be more far-reaching. For example, the cooling effect can extend further into the object having the treated surface. This can occur, for example, by cooling in the form of heat conduction and/or as a result of at least partial absorption of the cryogenic liquid. A corresponding absorption depends on the type of cryogenic liquid used and/or on the structural and chemical nature of the surface. For example, if more cryogenic liquid is applied locally, its cooling effect may last longer because a larger reservoir of cold is provided. In addition, by repeating or continuously continuing the application of the cryogenic liquid, the temperature profile over time at the surface and inside the impacted object, for example a substrate, can be controlled or regulated in conjunction with a suitable temperature measurement. Based on the temperature measurement, for example, the delivered amount of cryogenic liquid, such as the number and/or frequency of droplets, can be controlled. For example, the delivery of the cryogenic liquid can be controlled to reach and/or maintain a predefined temperature. A non-contact temperature measurement is preferred.

During application of the cryogenic liquid to the surface, a print head used for application may be in contact with or spaced from the surface.

According to embodiments, the digitally controlled application of the cryogenic liquid controls a position of the dropletwise application in 2D or 3D, a volume of the dropletwise application, and/or an angle of the dropletwise application relative to the surface. For example, the volume and/or angle may be position dependent.

Embodiments may have the advantage that the application of the cryogenic liquid can be controlled in a position-dependent manner. Here, the position of one or more print heads and/or one or more print nozzles of a print head relative to the surface can be detected and controlled. For example, the droplet-by-droplet application is performed depending on a position in 2D, i.e., depending on a position within a plane parallel to the surface. For example, the droplet-by-droplet application is performed as a function of a position 3D, i.e., as a function of a position in three dimensions above the surface. For example, the surface may include structures and the print head may additionally be moved in the z-direction perpendicular to the surface in addition to movements in the x-and y-directions parallel to the surface. The x, y and z directions are, for example, coordinates of a Cartesian coordinate system.

Furthermore, the volume of the applied cryogenic liquid can be controlled, in particular depending on the position. By means of a corresponding volume regulation, for example, the degree of the cooling effect, a temporal duration and/or a spatial extension of the cooling effect of the cryogenic liquid, in particular into the object having the surface, can be controlled. The volume of the applied cryogenic liquid can be controlled, for example, by controlling the number and/or size of the droplets of cryogenic liquid applied at the same position.

Angular control of the dropletwise application of the cryogenic liquid relative to the surface makes it possible, for example, in the case of a structured surface, to cover corresponding structural elements of the surface with cryogenic liquid with pinpoint accuracy. For example, in the case of elevations and/or recesses, side walls of the corresponding elevations and/or recesses in the surface can also be precisely covered with cryogenic liquid droplet by droplet. For example, it is possible to selectively cover corresponding side surfaces.

A digital control system is understood to be a computer-based control system using control commands which, for example, controls the droplet-by-droplet application of the cryogenic liquid in a position-dependent manner. In particular, the delivery position of the cryogenic liquid in 2D or 3D as well as the volume, i.e., the number of droplets and/or droplet size, and the angle of the droplet delivery can be controlled. For example, a rate at which the droplets are delivered can also be controlled.

Digital control of the droplet-by-droplet delivery of the cryogenic liquid may have the advantage that the amount of cryogenic liquid delivered can be precisely controlled, on a droplet-by-droplet basis, for example, on a picoliter scale. In the case of digital control, control is accomplished using digital control commands. Corresponding control commands translate, for example, a predefined distribution scheme for distributing the cryogenic liquid on and/or over the surface into instructions for controlling a printer unit applying the cryogenic liquid to the surface.

According to embodiments, the cryogenic liquid is stored in a container that is fluidically coupled to a digital print head. The digital printhead is digitally controlled for droplet-by-droplet application of the cryogenic liquid to the surface.

For example, the distribution scheme for distributing the cryogenic liquid on and/or over the surface may define where, when, how much cryogenic liquid to apply, and whether or not the applied cryogenic liquid should reach the surface. For example, the distribution scheme defines a spatial and/or temporal distribution of the cryogenic liquid. For example, the distribution scheme defines a spatially and/or temporally varying delivery amount of the cryogenic fluid. For example, the distribution scheme may define a distribution in which the delivery of the cryogenic fluid is modulated such that the cryogenic fluid reaches the surface at one or more locations while not reaching the surface at one or more other locations. Thus, for example, a distribution can be implemented which comprises both a direct and an indirect temperature influence on the surface in a locally targeted manner.

Embodiments may have the advantage that, for example, a printing unit may be provided having a digital print head which is digitally controlled and, depending on the control, dispenses and/or applies the cryogenic liquid droplet by droplet over the surface.

According to embodiments, the digital print head is movable relative to the surface.

Embodiments may have the advantage that by moving the digital print head in 2D or 3D, different dispensing positions may be adopted relative to the surface and the cryogenic liquid may be dispensed, impinged or applied to the surface depending on the respective dispensing position. According to alternative embodiments, an object comprising the surface may be movable in 2D and/or 3D relative to the print head. For example, both the print head and the object may be movable. For example, a detection unit is further provided that detects the relative position of the surface and the digital print head with respect to each other. For this purpose, for example, the object and/or the digital print head may have position markings which are detected by the corresponding detection unit. For example, the corresponding sensing unit may comprise an image sensor for capturing image data. For example, the corresponding detection unit can be configured for distance measurement by means of interferometry, in particular laser interferometry.

For example, the position of the digital printhead can be controlled to maintain a predefined minimum distance from the surface. For example, the position of the digital printhead may be controlled to maintain a constant distance from the surface and/or a distance within a predefined interval. For example, the position of the digital print head may be controlled such that the distance of the digital print head from the surface varies based on position. This can be advantageous, for example, for complex and/or jagged 3D structures of the surface.

According to embodiments, the surface has a temperature above −50° C., above −20° C., in particular above 0° C., prior to application of the liquid. According to embodiments, the surface is at room temperature prior to application of the liquid. According to embodiments, the surface has the body temperature of an animal or a human prior to application of the liquid.

According to embodiments, the surface has a temperature of 100° C. or less prior to application of the liquid.

Embodiments may have the advantage that strong local cooling of the surface can be achieved by means of the cryogenic liquid. For example, the temperature of the cryogenic liquid may be −275° C. to −75° C. For example, the cryogenic liquid can be −272° C. to −269° C. in the case of helium, −259° C. t −252° C. in the case of hydrogen, −210 to −196° C. in the case of nitrogen, 189 to −186° C. in the case of argon, −218° C. to −183° C. in the case of oxygen, and −78.5° C. in the case of carbon dioxide. The corresponding temperature ranges can be varied by adjusting the pressure. For example, at a lower pressure, lower temperatures can be obtained with the cryogenic liquid without causing a phase transition from liquid to solid. For example, at a higher pressure, higher temperatures can be achieved with the cryogenic liquid without a phase transition from liquid to gas occurring.

According to embodiments, the application of the cryogenic liquid is performed under a protective atmosphere. According to embodiments, the application of the cryogenic liquid takes place under a protective atmosphere with increased or decreased oxygen content relative to the normal atmosphere. For example, the use of a protective atmosphere serves to prevent chemical reactions between the surface exposed to the cryogenic liquid and the atmosphere, i.e., the protective atmosphere.

Embodiments may have the advantage of using a protective atmosphere to prevent undesirable chemical reactions of the cryogenic liquid and/or chemical components of the surface. For the protective atmosphere, for example, protective gases can be used, such as nitrogen, carbon dioxide, oxygen, argon, helium, hydrogen and/or carbon monoxide. The content of the corresponding protective gases in the atmosphere can be increased relative to the normal atmosphere. For example, a protective atmosphere comprising exclusively one or more protective gases may be used. For example, the protective atmosphere may comprise or consist exclusively of a protective gas identical to the cryogenic liquid(s) used. For example, the oxygen content in the atmosphere may be changed, and in particular an oxygen-free atmosphere may be used, such as in the case of using hydrogen as the cryogenic liquid. On the other hand, for example, the oxygen content in the protective atmosphere can be increased relative to the normal atmosphere. This can be particularly advantageous if the use of the cryogenic liquid is in connection with an oxidation process. In this case, for example, hydrogen is not used as the cryogenic liquid.

According to embodiments, the application of the cryogenic liquid is performed under positive pressure. According to embodiments, the cryogenic liquid is applied under negative pressure.

Embodiments may have the advantage that by controlling the pressure under which the cryogenic liquid is applied, the temperature of the applied cryogenic liquid can be controlled. This means that the physical state of the cryogenic liquid in the liquid phase is dependent on the pressure. By changing the pressure, the temperature at which the cryogenic liquid is actually in the liquid state can be controlled. Thus, the temperature that the cryogenic liquid exhibits when applied as a liquid can also be varied. For example, the pressure can be decreased and thus lower temperatures can be realized with the cryogenic liquid without it changing to the solid phase. For example, the pressure can be increased and thereby higher temperatures can be realized with the cryogenic liquid without it transitioning to the gas phase. Furthermore, by controlling the pressure of the atmosphere in which the cryogenic liquid is applied to the surface, it can be controlled whether the applied cryogenic liquid remains in the liquid phase or changes to a gaseous or solid phase. Thus, for example, contact times between the cryogenic liquid and the surface can be controlled. On the one hand, this makes it possible to control the effect of the filling effect and, on the other hand, interactions of the cryogenic liquid with further physical and/or chemical processes for which cooling is performed can thus be controlled and/or prevented. For example, the pressure can be reduced and thus a transition of the cryogenic liquid from the liquid to the gaseous phase can be achieved. Thus, the cryogenic liquid can detach from the surface again within a short time and a disturbance of further subsequent physical and/or chemical processes can be avoided.

According to embodiments, the cryogenic liquid is helium, hydrogen, nitrogen, argon, oxygen, carbon dioxide, or a mixture of one or more of these.

According to embodiments, the use further comprises:
Capture a 2D and/or 3D structure of the surface and/or an object comprising the surface,
determining structural elements of the surface to which the cryogenic liquid is to be applied as a function of the sensed 2D and/or 3D structure; and
generating control data adapted to control the application of the cryogenic liquid to the particular structural elements.

Embodiments may have the advantage of detecting a 2D and/or 3D structure of the surface or the object comprising the surface. A corresponding detection can, for example, be performed visually by means of a camera. For example, the detection may be performed by means of a microscope. In particular, for example, a scanning probe microscope, such as a scanning tunneling microscope, atomic force microscope, magnetic force microscope, optical scanning near-field microscope, or an acoustic scanning near-field microscope may be used. For example, a digital model, such as a 3D model, of the surface is created using the captured data. Based on the captured 2D and/or 3D surface structures, structural elements of the surface to which the cryogenic liquid is to be applied can be determined. A corresponding determination of the structural elements can be performed, for example, by a selection of a user or automatically using an image recognition method. For example, determining the structural elements of the surface and/or generating the control data is done automatically. For example, the control data is generated automatically as a function of the structural elements of the surface determined, for example, by means of an image recognition method.

For example, for a particular application, it may be specified which characteristic properties are exhibited by structural elements to which cryogenic liquid is to be applied. Corresponding properties can be, for example, geometric properties or physical properties, such as color in the case of image capture of the surface structure. Color here refers to the reflectance behavior under irradiation with light of one or more wavelengths in the visible and/or non-visible wavelength range, i.e., monochromatic or polychromatic light. Depending on the particular structural elements, control data may be generated that controls the application of the cryogenic liquid to the particular structural elements. For example, the corresponding control data can define at which positions which volumes of cryogenic liquid are to be dispensed or applied at which angle. For example, it is also possible to control when and at what rate cryogenic liquid is to be applied drop by drop at which positions.

According to embodiments, the use further comprises creating a digital distribution scheme for applying the cryogenic liquid to the determined structural elements in dependence on the detected 2D and/or 3D structure. The control data is configured to control the application of the cryogenic fluid to the determined structural elements in accordance with the distribution scheme.

For example, a digital distribution scheme is created that defines the application of the cryogenic liquid to the specific structural elements depending on the detected 2D and/or 3D structure. For this purpose, for example, a 3D model of the structures of the surface is used. A corresponding distribution scheme can be used, in particular, to visualize a proposal for applying the cryogenic liquid. For example, a corresponding proposal is generated automatically and/or using user input. For example, based on the captured 2D and/or 3D structure or a digital model of the surface generated using the captured data, it is possible to define at which positions cryogenic liquid is to be applied and at which angles. Furthermore, for example, 3D structures can be used to define which volume of cryogenic liquid is to be applied. For example, if an elevation is to be completely cooled, it may be necessary to apply a larger amount of cryogenic liquid, i.e., a larger volume, locally to the corresponding elevation than in the case of a planar structure where no cooling effect is required in depth, i.e., perpendicular to the surface into the corresponding object. For example, the control data is adapted to control the application of the cryogenic liquid to the particular structural elements according to the distribution scheme. In other words, the control data may be a translation of the distribution scheme into control data, i.e., control commands, for a controller controlling the application of the cryogenic liquid.

According to embodiments, the digital distribution scheme is displayed on a display device, and controlling the application of the cryogenic liquid using the control data according to the distribution scheme requires receiving a confirmation of the displayed distribution scheme via an input device.

Embodiments may have the advantage that the digital distribution scheme is displayed on the display device and thus visualized to a user. The user may examine the corresponding distribution scheme. For example, the corresponding distribution scheme is displayed together with an image and/or a digital model of the detected surface structure. The user may confirm the distribution scheme or specify corrections. For example, the user can change positions at which cryogenic liquid is to be applied droplet by droplet; for example, the user can change droplet size, droplet number, and/or droplet rate. Furthermore, for example, angles can be set under which the cryogenic liquid is applied. For example, a use of the control data according to the distribution scheme requires an explicit confirmation of the displayed distribution scheme by the user. Such confirmation may be received from the user directly in response to the display of the distribution scheme, or after receiving correction data and modifying the distribution scheme according to the received correction data.

According to embodiments, the surface is living or dead biological material. In particular, the biological material comprises: Microorganisms, a cell culture and/or a cell cluster, in particular an in-vivo or in-vitro cell cluster, in particular an in-vitro cell cluster for the growth of an artificial organ or organ part, wherein the biological material is in particular human or animal skin or a tissue sample.

Very rapid cooling of cells causes ice crystals to form inside and on the surface of cells, perforating the cell membrane. This leads to the penetration of water into the cell, the breakdown of vital regulatory processes and rapid cell death. The rate of freezing has been shown to have a strong effect on the proportion of cells that die due to freezing. Embodiments may have the advantage of having a cooling rate (“freezing rate”) in excess of 100° C. per minute while cooling the tissue to below −25° C., preferably to a temperature of −45° C. to −25° C. It has been shown that this causes a very high proportion of cells cooled in this way to die. This temperature range is particularly useful for applications in which the low temperatures are used for the targeted killing of cells in a spatially narrowly defined area.

In other embodiments, the amount of cryogenic fluid applied is dosed to cool the temperature of the cells to about 0.5° C. to 15° C. This results in local suppression of pain sensations and can be done, for example, in addition to local surgical, e.g., laser-based, procedures. For example, the cryogenic liquid can first be applied with the print head to a specific tissue to be operated on or treated with a laser, so that the tissue cools down considerably locally, but without the formation of ice crystals and cell death. However, the cooling leads to a suppression of the sensation of pain at this site, so that subsequent surgical treatment with a scalpel or laser, for example, is perceived as less painful. For example, the cryogenic liquid can be applied additionally or exclusively to the outer edges of the tissue area that will later be removed by scalpel or laser or has already been removed in a previous treatment step.

For example, a control device may control the amount of cryogenic fluid delivered via the printhead per unit time and/or the position of the printhead relative to the surface such that the amount and frequency of droplets applied ensures that the temperature of the tissue surface and/or cells within an in vivo or in vitro tissue is maintained within a predefined temperature range for a predefined period of time. The temperature range and the depth profile of the temperature range depend on the particular application (cryogenic destruction of cells or pain reduction, depth and 3D structure of subcutaneous structures (e.g. warts, cancerous ulcers, etc.).

According to embodiments, the process is repeatedly applied to the same surface multiple times, e.g., twice or three times, e.g., multiple times within an hour or multiple times within a second. This may further increase the proportion of dead and/or thermally inactivated cells.

According to embodiments, the control device is configured to control the application of droplets of the cryogenic liquid to the surface such that the application is continuous and maintains individual areas on and below the surface at a defined temperature for a defined period of time.

According to one embodiment, the apparatus has multiple reservoirs for multiple cryogenic fluid volumes. The print head has a plurality of nozzles, each of which is fluidly connected to one of the containers. The containers include different cryogenic fluids and/or cryogenic fluids of different temperatures. This makes it possible, similar to inkjet printing, to very finely granularly cool surface areas and/or tissue areas down to a specific temperature by complex control of the print patterns generated by the individual print nozzles.

For example, the printing process can be controlled in such a way that a first cryogenic liquid from a first container with a particularly low temperature is applied in a high drop density from a first nozzle to a first area of the surface under which the center of the tissue to be destroyed (e.g. wart, cancerous tumor, etc.) is located. The tissue is cooled to such an extent that the cells die by perforation of the cell walls by ice crystals. In addition, a second cryogenic liquid is applied from a second container at a not-quite-as-low temperature and/or with lower droplet density to surrounding, “second” areas of the surface. The surrounding skin and tissue areas are cooled here, but to temperatures above 0° C. to avoid ice formation.

In addition or alternatively, the device has several nozzles that can be individually controlled to deliver droplets of the cryogenic liquid of different sizes and/or different frequencies. This also makes it possible to cool down surface areas and/or tissue areas to a specific temperature in a very fine granular manner by complex control of the pressure patterns generated by the individual pressure nozzles.

For example, the printing process can be controlled so that a first nozzle applies the cryogenic liquid in high drop density and/or with a large drop volume to a first area of the surface under which the center of the tissue to be destroyed is located. The tissue is cooled to such an extent that the cells die by perforation of the cell walls by ice crystals. In addition, a second nozzle applies the cryogenic liquid in low drop density and/or small drop volume to surrounding, “second” areas of the surface. The surrounding skin and tissue areas are cooled here, but to temperatures above 0° C. to avoid ice formation.

“Small” or “large/high” drop density or volumes can be a relative indication, including, for example, that a “small” drop has a volume at least 20% smaller than a “large” drop, or that a “small” drop density is at least 20% less than a “large” drop density.

This can be advantageous because cryobased destruction of tissue and cryobased nondestructive cooling of surrounding tissue for the purpose of pain reduction can be accomplished in a single application step. In some cases, low temperatures are already used for pain relief during surgical procedures (e.g., with scalpels or lasers). However, these procedures have the disadvantage of requiring a high level of equipment (additional cryogenic equipment is required in addition to surgical equipment). In contrast, embodiments of the invention may have the advantage that the surgical procedure and the cooling for pain relief can be performed in a single step and with the same device. This also has the advantage of improved spatial coordination between the cell-destructive, cryosurgical procedure and a purely “cell-sedating”, pain-relieving treatment of the surrounding tissue. This is because both cell destruction and sedative cooling are controlled by the same device and the same control device.

The term “cryogenic liquid” is used here to describe a liquid that is used to cool objects by applying this liquid, e.g., in technical applications or scientific experiments. Typically, a cryogenic liquid has a temperature below 0° C. For example, the cryogenic liquid may be a liquid gas produced, e.g., by central facilities or commercial suppliers by liquefaction of the corresponding gases (for air/nitrogen, e.g., by the Linde process) and brought to the respective application by special transport containers. The liquid gas may be, for example, liquid nitrogen and/or liquid helium, hydrogen, nitrogen, argon, oxygen or mixtures of two or more of the aforementioned gases.

The cryogenic liquid used for biological, medical and/or cosmetic purposes may be, in particular, liquid nitrogen or other non-toxic, biocompatible cryogenic liquids.

The use of cryogenic liquids makes it possible to achieve very high freezing rates and is less dangerous to handle (risk of explosion and fire) than liquid oxygen, for example.

According to embodiments, the printhead is designed as a matrix printhead in which an application pattern or print image is generated by the targeted firing or deflection of small droplets of liquid. The printhead may be designed, for example, as a drop-on-demand printhead, i.e., as a single droplet firing printhead.

According to embodiments, the printhead is configured to generate droplets of a droplet size of less than 100 picoliters (pl), preferably less than 10 picoliters. The generation of such fine droplets may have the advantage of producing a particularly fine-textured pattern. Top devices are in the range of 1 pl to 5 pl. For example, the droplets may be sprayed at a frequency of over 10,000 droplets/second. According to embodiments, the print head includes a plurality of print nozzles that can deliver the droplets at a nozzle-individual frequency, wherein the nozzles can be individually controlled and can generate droplets at different frequencies. The generation of very small droplets in high and preferably variably adjustable

According to embodiments, the use is a method for the medical and/or aesthetic treatment of skin lesions. The skin lesions may be one or more of the following skin lesions:
Pigment spots,
Scar tissue, esp. overshooting scar tissue (keloid),
Warts, especially style warts and dell warts (Molluscum contagiosum),
Fibroma molle tumors,
Lentigo solaris lesions,
Hemangiomas,
Fire marks,
Keratoses, especially seborrheic keratosis;
skin cancer-related skin lesions.

For example, the removal of pigment spots, scar tissue, port-wine stains, warts, and/or keratoses may serve aesthetic purposes.

According to embodiments, the use is:
a cryosurgical procedure, in particular a local dermatological procedure; and/or
an interventional therapy procedure of tumors, e.g. therapy of liver metastases, lung or prostate tumors;
a procedure to temporarily or permanently inactivate or destroy nerve cells, e.g., pain therapy for phantom limb pain or to reduce pain from surgical procedures.

For example, temporary inactivation of neurons to reduce surgery-related pain can be performed before, during, or after surgery. Preferably, the surgery is a cryosurgical procedure performed by the same device that performs the cryogenic fluid pain management procedure. Preferably, the cryosurgical procedure and the pain management procedure are controlled and performed together in a single step.

A cryogenic jet of at least 5 milliseconds of liquid nitrogen can already provide significant pain relief.

According to embodiments, the surface is a human or animal skin in vivo or in vitro. The term “in vivo” is used here to describe processes that take place in a living organism. In contrast, processes that take place in an artificial environment (e.g., in a test tube) or generally outside living organisms are referred to as “in vitro”.

“In vitro” skin refers to two-dimensional or three-dimensional cell assemblies consisting of or containing skin cells, which are cultured on nutrient media or nutrient substrates using various methods. More generally, “in vitro” tissues, such as in vitro organs or organ parts, are those that are cultured outside a human or animal organism in an artificial environment. By regularly “printing” surface areas of this cell cluster with cryogenic liquid, the growth of the cells can be controlled locally in a defined manner. Areas of the cell cluster that have been regularly cooled grow more slowly, while cell death occurs in the case of strong cooling. Thus, the growth of skin cells in 2D and/or 3D can be influenced locally in a targeted manner, thereby giving the tissue grown (e.g., for transplantation purposes) a certain shape.

Growth control of “in vitro” skin and other “in vitro” cell assemblies can be beneficial for several reasons:

On the one hand, this enables very fine-grained local growth control. Growth-inhibiting agents can hardly be applied locally, as they diffuse in the medium. A temporal control of cell inhibition is also possible by the cryogenic liquid, because it can be terminated or prolonged at any time. Growth-inhibiting substances, on the other hand, can no longer be removed from the cell culture medium.

In addition, it is a digitally controlled, fully automated printing process that can be performed fully automatically, regularly and under sterile conditions. For example, the printing device can be completely integrated into a sterile incubator for cell cultures, so that it is not necessary to open the incubator to perform manual steps that manipulate cell growth.

The “in vitro” synthesis of skin and other organs is a field with very great medical potential. For some cell types it could be shown that they grow on a matrix (e.g., cartilage cells on a matrix of jellyfish collagen). The matrix makes it possible to determine the 3D structure of the cells. However, this is not possible for all cell types. However, selective inhibition of cell growth by reducing temperature appears to be universally applicable to all cell types, since growth inhibition is at least partially attributable to a temperature-induced slowing of biochemical processes and chemical reactions in the cell.

According to embodiments, the surface is a substrate. One or more patterning steps are performed to pattern the surface and/or apply a two-dimensional or three-dimensional pattern to the surface. For example, the application of the cryogenic liquid occurs at least prior to one of the patterning steps to affect the subsequent patterning step. For example, the application of the cryogenic liquid results in a structuring step.

Embodiments may have the advantage that by using the cryogenic liquid, structuring processes of a substrate can be controlled locally. For example, by applying the cryogenic liquid, the surface temperature can be varied locally with pinpoint accuracy. Physical and/or chemical processes affecting the surface can depend on the corresponding surface temperature. For example, chemical reaction rates can be controlled. For example, a deposition rate can be temperature-dependent and thus a structure formation on the surface can be controlled. For example, a chemical ablation process, such as an etching process, may be temperature dependent and thus control of the ablation rate may be accomplished by controlling the local temperature. For example, a synthesis reaction in the course of polymerization, i.e., a synthesis reaction that converts like or unlike monomers into polymers, may be temperature dependent. A corresponding polymerization process can be controlled locally by a local temperature variation.

According to embodiments, the patterning step is a chemical reaction, a physical deposition step, and/or a polymerization step.

According to embodiments, the application of the cryogenic liquid locally controls a reaction rate of the chemical reaction.

According to embodiments, the application of the cryogenic liquid locally changes an aggregate state of a substance involved in the chemical reaction, physical deposition step, and/or polymerization.

Aggregate states can be varied locally by means of a local temperature variation. For example, substances involved can be converted from a gas phase to a liquid or solid phase by cooling. A corresponding change in aggregate state can influence a chemical reaction, a physical deposition step, and/or polymerization. For example, a contact of the corresponding substances with the surface can be established, increased and/or contact times or contact rates can be controlled by a phase transition.

According to embodiments, the surface is a substrate for a printed circuit.

Embodiments may have the advantage that the cryogenic liquid can be used, for example, for structuring a substrate on which a circuit is to be printed. The structuring can create a structural condition necessary for the printing or the geometric design of the circuit.

According to embodiments, the cryogenic liquid is used to manufacture a component. The component is, for example, an electronic component. The electronic component is, for example, a silicon-based and/or germanium-based or polymer electronic integrated circuit.

The device may be, for example, an electronic device, a mechanical device, and/or a device that provides a particular surface structure. Such a surface structure may serve, for example, to support, inhibit, and/or control a mechanical, electromagnetic, or chemical process. Mechanically, the surface structure may, for example, support, inhibit, and/or control mechanical motion, such as of atoms, molecules, or motion elements, on the surface. Electromagnetically, the surface structure may, for example, electromagnetically support, inhibit, and/or control interactions on or across the surface. Chemically, the surface structure can, for example, support, prevent and/or control a chemical process, for example as a catalyst or inhibitor.

According to embodiments, the application of the cryogenic liquid occurs prior to a doping step to locally modulate doping.

Embodiments may have the advantage that doping can be locally modulated by local temperature control using the cryogenic liquid. For example, doping can be accomplished by diffusion. Diffusion is a thermally activated equalization process of a concentration difference, for example in a solid. In the case of an existing concentration difference, foreign atoms can penetrate into another solid body at sufficiently high temperatures and move there. The corresponding movement of the foreign atoms is based, for example, on Brownian molecular motions. By means of a local temperature variation, in particular temperature reduction by applying the cryogenic liquid, the temperature and thus the movement of molecules or atoms can be locally restricted. Thus, for example, a doping rate can be lowered locally.

In a further aspect, the invention relates to an apparatus configured to carry out the use of a cryogenic liquid according to any of the embodiments described herein. The apparatus is configured, for example, for one or more or any of the aforementioned embodiments of a use of the cryogenic liquid.

In another aspect, the invention relates to an apparatus for applying a cryogenic liquid to a surface to affect a temperature of the surface, comprising a container for holding the cryogenic liquid, a digital printhead fluidically connected to the container, and a control device for controlling the printhead to apply the cryogenic liquid to the surface in droplets.

Embodiments can have the advantage that a corresponding device can be provided, for example in the form of a printing unit, which prints the cryogenic liquid dropletwise onto the surface by means of the digital print head. The droplet-by-droplet application is thereby controlled by a control device. For control, the corresponding control device uses, for example, control data. The corresponding control data may, for example, result from a distribution scheme provided for the application of the cryogenic liquid. In other words, the control data may translate the corresponding distribution scheme into control commands with which the control device controls the printer unit. The control device may be a device integrated into the printer unit or, for example, a computer system to which the printer unit is connected.

According to embodiments, the printhead includes a plurality of individually controllable nozzles. Each of the nozzles is configured to control the size and/or frequency of the droplets ejected from the respective nozzle in response to control data from the control device.

According to embodiments, the device is a medical device, in particular a medical device for selectively applying the cryogenic fluid to an organ or organ part. The organ or organ part may be an in vivo or in vitro organ or organ part. In particular, the organ may be human or animal skin. The medical device may also be a device for selectively applying the cryogenic fluid to individual cells, in particular nerve cells.

The human skin is divided into three main layers: Epidermis (epidermis), Dermis (dermis, lat. corium) and Subcutis (hypodermis). The epidermis is a multi-layered keratinizing squamous epithelium that is usually between 0.03 and 0.05 millimeters thick. On the palms of the hands and the soles of the feet, the horny layer is up to several millimeters thick (“callus”). The dermis consists primarily of connective tissue fibers and serves to nourish and anchor the epidermis. It contains blood vessels, nerves, and smooth muscle and blood vessels important for temperature regulation. The hypodermis contains the larger blood vessels and nerves for the upper layers of the skin, as well as subcutaneous fat and loose connective tissue. Sensory cells for strong pressure stimuli, for example the lamellar corpuscles, are located in the subcutis.

By appropriate control of the print head, all skin layers can be cooled to temperatures below 10° C., also below 0° C., also below −20° C., also below −40° C. for a predefined duration. This can destroy tissue and/or reduce the activity of the nerves therein, especially for the purpose of pain relief).

According to embodiments, the device may be formed as a tool having a shaping that enables guiding and positioning of the print head with one hand. In particular, the shaping may be in the form of a pen, painting or drawing implement.

For example, the device may have an elongated shape and the print head may be located at the front end of the device. Preferably, the device has a retaining area configured to allow the device to be received and guided by a human hand. For example, the holding portion may have a recess or taper and/or be made of resilient material.

Additionally, or alternatively, the device may be an endoscope. For example, the device may comprise a first unit, which includes the container and the control device, and a second unit, which is intended for insertion into the body in order to destroy e.g. metastases or other unwanted tissue formations there and/or to inactivate individual nerves temporarily or permanently. The second unit includes the print head and optionally further elements (camera, temperature sensor, possibly surgical forceps, laser, etc.) and is coupled to the first unit via a preferably flexible connection.

For example, the device may be an endoscope with a camera. The camera is mounted at the same end of the device as the printhead and is oriented such that the camera is adapted to capture images of the printhead, and the surface contacted by the printhead. This can be advantageous as it provides the treating user, such as a physician, with a visual inspection and feedback signal regarding the printing process with the cryogenic fluid.

According to another embodiment, the device is a printing device that is an integral part of an apparatus for automated cultivation of one or more cell cultures.

According to another embodiment, the device is an apparatus designed to be movable in two or three dimensions for applying the cryogenic liquid to the individual cells and/or the organ or organ part, in particular an organ or organ part cultivated in vitro. According to embodiments of the invention, the apparatus includes a temperature sensor and is configured to sense the surface temperature of the organ or organ part during application using the temperature sensor and to use the surface temperature to control the application. For example, the number, size, and/or exit velocity of the cryogenic droplets and/or the distance or angle of the print head relative to the surface can be adjusted so that the temperature of the surface is within a predefined temperature range. This very fine-grained control of the surface temperature can be particularly advantageous when impacting biological material, as temperatures that are too low can lead to the destruction of individual cells or entire tissues. The death of cells should be avoided in application scenarios in which the aim is merely to slow down cell growth in a locally targeted manner or to alleviate pain. But also in applications in which specific tissue areas are to be destroyed, a narrow focus of the application area is advantageous, as damage to the surrounding tissue can be prevented.

For example, in some embodiments, the device may be configured to set the temperature at the surface within a range that slows cell growth but does not cause cell death. In particular, the device may be configurable to ensure that when a cryogenic fluid is applied to the tissue or organ or organ part, the surface temperature is within a set point temperature, the set point temperature being selected to avoid tissue damage and cell death. For example, by means of such a set temperature, the attainment of which is continuously checked via the—preferably non-contact—temperature sensor, the application process can be controlled in such a way that the cryogenic droplets evaporate before reaching the surface of the cells or tissue, so that there is no direct contact with the cell, but cooling takes place indirectly via the air. Cell death induced by direct contact with cryogenic liquid droplets can thus be avoided.

The other embodiments of a device described here for the application of a wide variety of biological and non-biological materials can also be equipped with a—preferably non-contact—temperature sensor and/or can be configured to carry out the application in such a way that the temperature of the applied surface lies within a target temperature range that excludes damage to the material. Such a temperature range can, for example, be a temperature range at which a cooling effect only occurs via the ambient air because the droplets have evaporated before reaching the surface.

In another aspect, the invention relates to a system for manufacturing a device, in particular an electronic device, comprising an apparatus according to any of the embodiments described above. The surface is a substrate. In the course of manufacturing the device, the apparatus performs one or more patterning steps to pattern the surface and/or apply a two-dimensional or three-dimensional pattern to the surface. For example, the application of the cryogenic liquid by the apparatus occurs at least prior to one of the patterning steps to affect the subsequent patterning step. For example, the application of the cryogenic liquid results in a patterning step. The device may be, for example, an electronic device, a mechanical device, and/or a device that provides a particular surface structure. Such a surface structure may serve, for example, to support, inhibit, and/or control a mechanical, electromagnetic, or chemical process. Mechanically, the surface structure may, for example, support, inhibit, and/or control mechanical motion, such as of atoms, molecules, or motion elements, on the surface. Electromagnetically, the surface structure can, for example, electromagnetically support, inhibit, and/or control interactions on or across the surface. Chemically, the surface structure can, for example, support, prevent and/or control a chemical process, for example as a catalyst or inhibitor.

Embodiments may have the advantage that the cryogenic liquid can be used to control physical and/or chemical processes in the course of a fabrication of a device, such as an electronic device. For example, a system for manufacturing a corresponding device comprises a corresponding printing unit with which the cryogenic liquid can be applied dropletwise to a surface of a substrate or to a substrate. The application can, for example, take place simultaneously with further structuring steps. In this case, for example, a printing unit and a corresponding structuring unit, which structures the surface by means of chemical and/or physical processes, are integrated into one unit. Alternatively, application of the cryogenic liquid can take place, for example, before a structuring step. In this case, a printer unit can be provided, for example, which prepares the surface accordingly by applying the cryogenic liquid. Structuring then takes place in an adjacent structuring unit, into which the treated surface is transferred.

In the following, embodiments of the invention are explained in more detail with reference to the drawings. Showing:

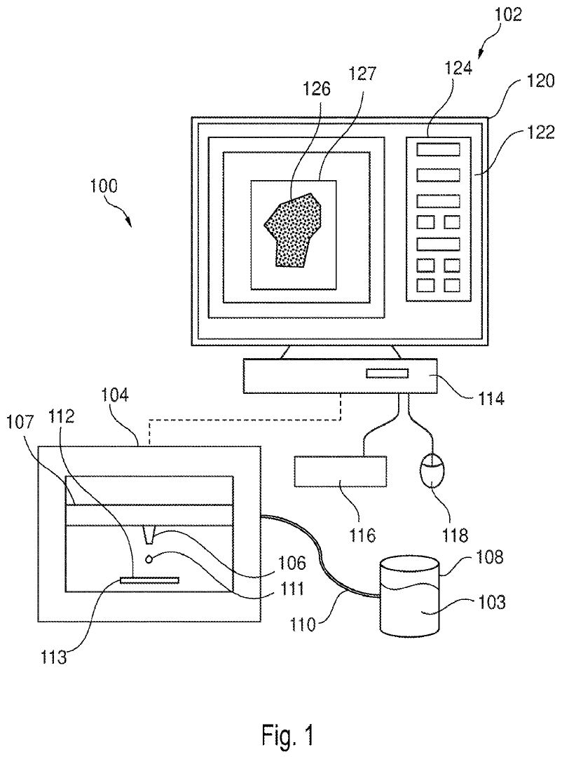

FIG. 1 a schematic view of an exemplary device for the application of a cryogenic liquid;

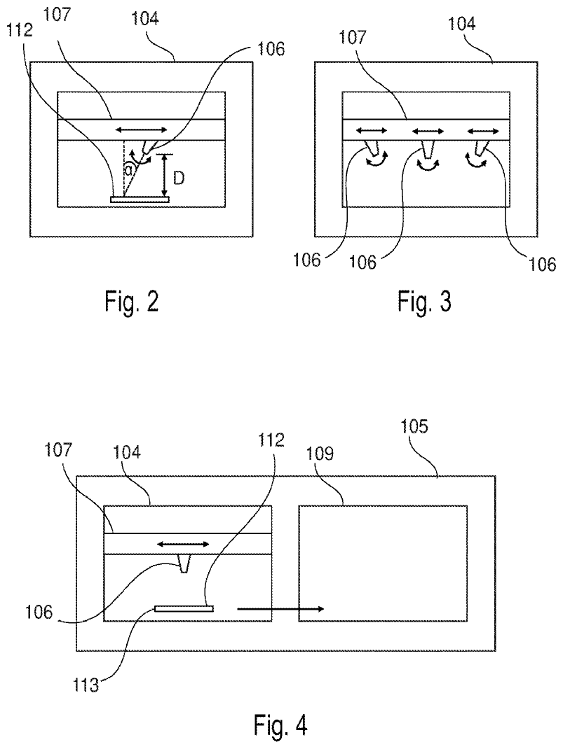

FIG. 2 a schematic view of an exemplary printer unit for the application of a cryogenic liquid;

FIG. 3 a schematic view of an exemplary printer unit for the application of a cryogenic liquid;

FIG. 4 a schematic view of an exemplary system for manufacturing a component;

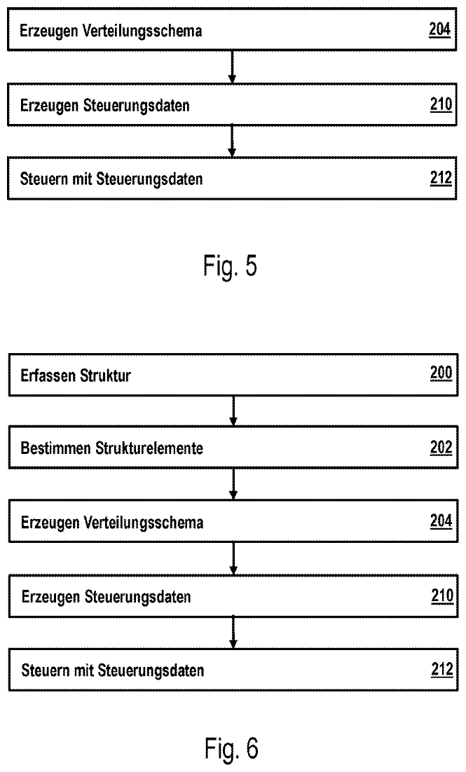

FIG. 5 a flow diagram of an exemplary process for applying a cryogenic liquid;

FIG. 6 a flow diagram of an exemplary process for applying a cryogenic liquid;

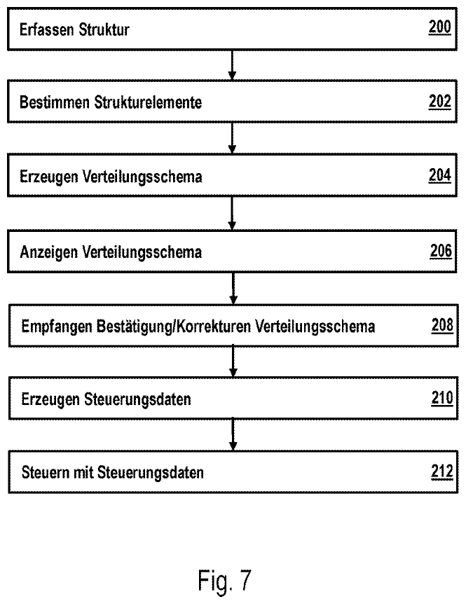

FIG. 7 a flow diagram of an exemplary process for applying a cryogenic liquid;

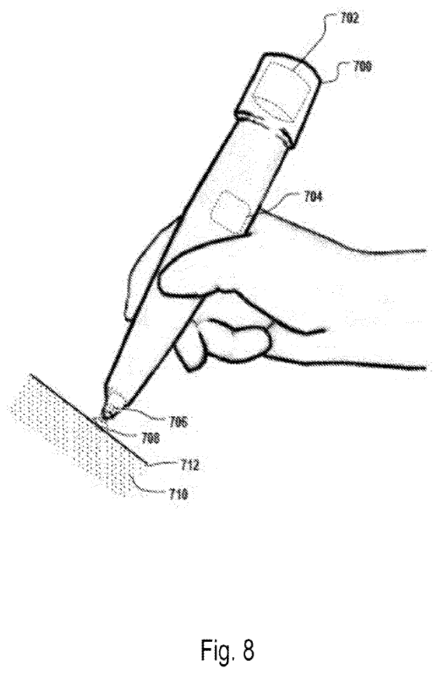

FIG. 8 a schematic view of an exemplary device for cryosurgical application of a cryogenic liquid;

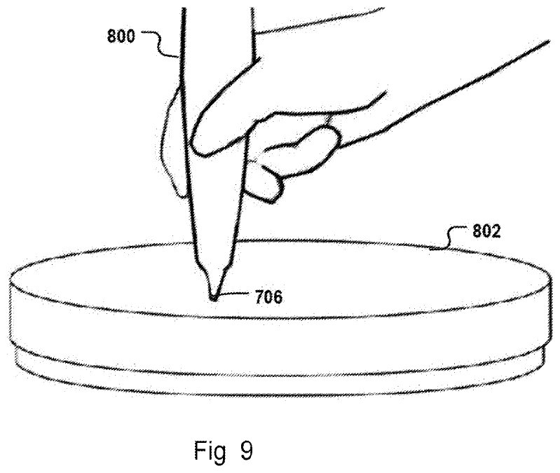

FIG. 9 a schematic view of an exemplary device for manual application of a cryogenic liquid in vitro; and

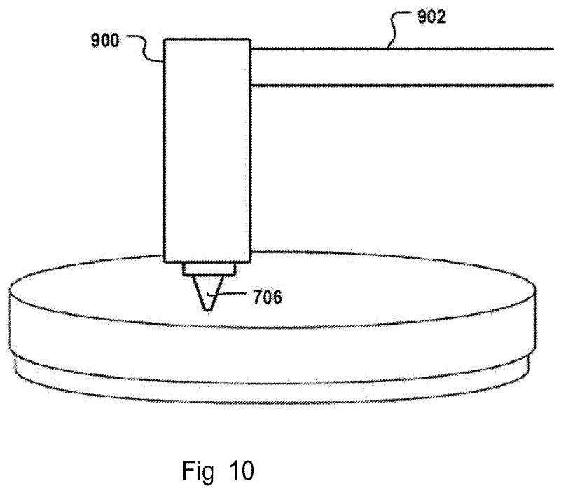

FIG. 10 a schematic view of an exemplary device for the automatic application of a cryogenic liquid in vitro.

Elements of the following embodiments which correspond to each other or are identical are each identified with the same reference signs.

FIG. 1 shows an exemplary device 100 for applying a cryogenic liquid 103 dropletwise to a surface 112 of an object 113. For example, a printer unit 104 is provided comprising a printhead 106 configured to apply droplets 111 of cryogenic liquid 103 to the surface 112. To apply the cryogenic liquid 103, a corresponding object 113 is placed, for example, in the printer unit 104. The printer unit 104 is fluidically connected by means of a fluidic line 110, for example, to a container 108 in which the cryogenic liquid 103 to be applied is located. One or more printheads 106 can be provided, for example, which can be moved in two or three spatial directions by a motion unit 107. The printheads 106 may each comprise, for example, one or more individually controllable nozzles. Each of the nozzles may be configured, for example, to control the size and/or frequency of droplets ejected from the respective nozzle in response to control commands from a control unit 102. For example, a protective atmosphere may be present within the printer unit 104. Further, the pressure within the printer unit 104 may be increased or decreased relative to the normal pressure of the environment.

The control unit 102 may be, for example, a computer system or computing device. The corresponding computing device may include hardware components 114 having one or more processors and a memory in which program instructions are stored for controlling the control unit 102 and the printer unit 104. For receiving input, the control unit 102 may include input devices, such as a keyboard 116 and/or a mouse 118. Further, the control device may include a display device, such as a screen 120, on which a graphical user interface or GUI (“Graphical User Interface”) is displayed. The corresponding graphical user interface 122 may display, for example, an image 127 of the surface 112, as well as a distribution scheme 126 indicating a proposed distribution of the cryogenic fluid on and/or over the surface 112. For example, to confirm a proposed distribution scheme 126 and/or to correct the corresponding proposal, the graphical user interface 122 may include control elements 124. By means of the corresponding control elements 124, a user may confirm the proposed distribution scheme 126 and/or make corrections to the proposed distribution scheme 126 using the input devices 116, 118. For example, to capture the image 127 of the surface 112, the device 100 may further comprise a capture device. The corresponding capture device may be, for example, a visual capture device, such as a camera. In particular, the capture device may be a microscope. The image 127 of the surface 112 may be used to determine structural elements to which the cryogenic liquid 103 is to be applied. For example, a distribution scheme 126 based on image recognition of pre-existing structures of the surface 112 may be determined using the image 127. Alternatively, a patterning scheme for patterning the surface 112 may be predetermined, which is transferred to the image 127, and a distribution scheme 126 of the cryogenic liquid may be generated based on the designated patterning scheme.

FIG. 2 shows a detailed view of an exemplary printer unit 104 of FIG. 1. The corresponding printer unit 104 comprises a printhead 106. The corresponding printhead may comprise one or more print nozzles. A position of the corresponding print head relative to the surface 112 may be controlled, for example, by a motion unit 107. The corresponding motion unit 107 allows, for example, the printhead 106 to be moved relative to the surface 112 in 2D, i.e., in a plane parallel to the surface 112, and/or in 3D. Thus, for example, in the case of a traverse in 3D, a distance D of the printhead 106 from the surface 112 can be adjusted. For example, the corresponding distance D may be varied, in particular as a function of a 2D position relative to the surface 112. Further, the distance D may be kept constant and/or kept constant within a predetermined interval. Further, a minimum distance between the printhead 106 and the surface 112 can be maintained, for example, by regulating the distance D. Further, an angle a at which the print head 106 applies the cryogenic liquid dropletwise to the surface 112 can be varied. A corresponding variation of the angle a can be achieved, for example, by pivoting the printhead 106 about one, two or three vertically stacked pivot axes.

For example, a protective atmosphere may be created within the printer unit 104 and/or the prevailing pressure may be regulated. By using a protective atmosphere and/or regulating the pressure under which the cryogenic liquid is applied to the surface 112, the effect of the cryogenic liquid, in particular the effect on the surface and/or on substances involved in physical and/or chemical reactions, can be controlled locally in a targeted manner. Furthermore, the effect of the substances involved in the physical and/or chemical reactions can be controlled by using a protective atmosphere and/or regulating the pressure.

FIG. 3 shows another exemplary embodiment of a printer unit 104 comprising a plurality of printheads 106. The motion unit 107 may be designed, for example, to allow the individual printheads 106 to be moved independently of each other in 2D and/or 3D. Further, the respective print heads may be independently pivoted about, for example, one, two, and/or three pivot axes that are perpendicular to each other.

FIG. 4 shows an apparatus 105 for manufacturing a component, in particular an electronic component. For this purpose, for example, an object 113 is provided, the surface 112 of which is a substrate that is to be structured for manufacturing the component. Cryogenic liquid is applied to the surface 112 by means of the printhead 106 for local control of the patterning process or the physical and/or chemical processes involved in the patterning process. The application of the cryogenic liquid may, for example, occur simultaneously with a patterning of the surface 112. In this case, the printer unit 104 would be integrated into the structuring unit 109. Alternatively, as shown in FIG. 4, the application of the cryogenic liquid may occur prior to a corresponding patterning step. In this case, for example, the cryogenic liquid is applied to the surface 112 of the object 113 by means of the printhead 106 that can be positioned using the motion unit 107. The application may be performed, for example, according to a previously defined distribution scheme. Subsequently, the corresponding object 113 is transferred to the structuring unit 109. For example, the same atmospheres, in particular a protective atmosphere, prevail in the printer unit 104 of the structuring unit 109. Furthermore, identical pressures may prevail in each of the two units 104, 109. Alternatively, the pressures prevailing in the units 104, 109 could differ.

The structuring unit 109 may, for example, be a unit configured to carry out a chemical reaction, a physical deposition step, and/or a polymerization step. For this purpose, the structuring unit 109 provides, for example, the substances involved in the corresponding structuring step and controls the physical framework conditions. A local temperature variation to influence the structuring step is then performed, for example, by the local distribution of the cryogenic liquid.

FIG. 5 shows a flowchart of an exemplary process for applying a cryogenic liquid to a surface. In block 204, a distribution scheme is generated. For example, a corresponding distribution scheme may be specified or set by a user. Alternatively, a distribution scheme may be based on a predefined structure of the surface to be produced to which the cryogenic liquid is to be applied. For example, a corresponding patterning template is provided that defines the predefined structure to be fabricated. For example, the distribution scheme may be a distribution of the cryogenic liquid according to the predefined structural shapes. For example, the distribution scheme for distributing the cryogenic liquid on and/or over the surface may define where, when, how much cryogenic liquid to apply, and whether or not the applied cryogenic liquid should reach the surface. For example, the distribution scheme defines a spatial and/or temporal distribution of the cryogenic liquid. For example, the distribution scheme defines a spatially and/or temporally varying delivery amount of the cryogenic fluid. For example, the distribution scheme may define a distribution in which the delivery of the cryogenic fluid is modulated such that the cryogenic fluid reaches the surface at one or more locations while not reaching the surface at one or more other locations. Thus, for example, a distribution can be implemented which comprises both a direct and an indirect temperature influence on the surface in a locally targeted manner.

At block 210, control data is generated using the distribution scheme. The corresponding control data is, for example, control commands for controlling one or more print heads to apply the cryogenic liquid to a surface. For example, the control data may define at which positions of the print head relative to the surface, how much cryogenic liquid to apply, and at what angle. At block 212, a corresponding device, such as a printing unit, is controlled to apply the cryogenic liquid to the surface using the control data. In other words, a dispensing of the cryogenic liquid over the surface and/or an application of the cryogenic liquid on the surface is performed according to the distribution scheme generated in block 204. Following or simultaneously with the application of the cryogenic liquid, for example, further physical and/or chemical processes can be carried out in a controlled manner, which are influenced by the local temperature change caused by the cryogenic liquid.

FIG. 6 shows a flow chart of an exemplary process for applying a cryogenic liquid, which in addition to the process shown in FIG. 5 comprises a detection of a structure of the surface in block 200. A corresponding detection of a structure of the surface can be advantageous, for example, if the physical and/or chemical processes which are to be influenced by the application of the cryogenic liquid depend on predetermined structures of the surface. In this case, for example, the structure of the surface is detected in block 200. In block 202, structural elements to which the cryogenic liquid is to be applied are determined. The corresponding determination of the structural elements can be performed, for example, by input from a user and/or can be performed automatically. In the case of automatic determination of the structural elements, for example, an image recognition method may be used. For example, the corresponding structural elements may be determined based on their geometry and/or their visual appearance, such as their color. For example, the distribution scheme in block 204 is generated based on the determined structural elements such that the positions at which the cryogenic fluids are applied to the surface correspond to the positions of the structural elements. Further, for example, the amounts of cryogenic liquid applied may be determined as a function of the structural elements, for example as a function of the geometric dimensions of the determined structural elements. Further, for example, the angle at which the cryogenic liquid is to be applied may be varied as a function of the geometry of the structural elements. In block 210, control data is generated using the distribution scheme, which is used in block 212 to control a corresponding device for applying the cryogenic liquid. Following or simultaneously with the application of the cryogenic liquid, for example, further physical and/or chemical processes can be carried out in a controlled manner, which are influenced by the local temperature change caused by the cryogenic liquid.

FIG. 7 shows a flow chart of another exemplary process for applying a cryogenic liquid to a surface. As in the process according to FIG. 6, the structure of a surface to which the cryogenic liquid is to be applied is first determined in block 200. In block 202, structural elements are determined which are to be at least partially covered with the cryogenic liquid. In block 204, a distribution scheme for applying the cryogenic liquid is generated using the determined structural elements. In block 206, the generated distribution scheme is displayed, for example, on a display device. At block 208, a user's confirmation of the distribution scheme is received. Alternatively, corrections to the distribution scheme may be received and the displayed distribution scheme corrected accordingly. For example, after the corrections are completed, the appropriately corrected distribution scheme is confirmed. In block 210, the prerequisite of which is, for example, confirmation of the distribution scheme, control data for controlling a device for applying the cryogenic liquid in accordance with the distribution scheme is generated. Finally, in block 212, control of the corresponding device is performed using the control data generated in block 210. Simultaneously and/or subsequently to the application of the cryogenic liquid, one or more further physical and/or chemical processes can take place in a controlled manner, which are locally controlled by the local temperature control by means of the cryogenic liquid.

FIG. 8 shows an exemplary device 700 for applying a cryogenic liquid 708 to a surface 712 for use in a medical or cosmetic context. For example, the device may be used to destroy or remove skin lesions. The device includes a container 702 for holding the cryogenic liquid, a digital printhead 706 fluidly coupled to the container, and a control unit 704 for controlling the print head to apply the cryogenic liquid to the surface in droplets.

Thus, the device 700 may be a medical device, particularly a medical device for selectively applying the cryogenic fluid to an organ or organ part. For example, the device may be used for cryosurgical medical or cosmetic applications.

The surface 712 may be, for example, the epidermis of human or animal skin 710 or any other surface of individual cells or a cell assembly.

The human skin is divided into three main layers: Epidermis (epidermis), Dermis (dermis, lat. corium) and Subcutis (hypodermis). The epidermis is a multi-layered keratinizing squamous epithelium that is usually between 0.03 and 0.05 millimeters thick. On the palms of the hands and the soles of the feet, the horny layer is up to several millimeters thick (“callus”). The dermis consists primarily of connective tissue fibers and serves to nourish and anchor the epidermis. It contains blood vessels, nerves, and smooth muscle and blood vessels important for temperature regulation. The hypodermis contains the larger blood vessels and nerves for the upper layers of skin, as well as subcutaneous fat and loose connective tissue. Sensory cells for strong pressure stimuli, for example the lamellar corpuscles, are located in the subcutis.

By appropriate control of the print head, all skin layers can be cooled to temperatures below 10° C., also below 0° C., also below −20° C., also below −40° C. for a predefined duration. This can destroy tissue and/or reduce the activity of the nerves therein, especially for the purpose of pain relief before, during or after local surgery.

Preferably, the cryogenic liquid is not applied according to the “all or nothing” principle, but via a plurality of nozzles which can be individually controlled and which eject drops of the cryogenic liquid in such a way that the droplet ejection of the various nozzles differs in terms of size and/or frequency. This can create complex patterns that are individually adapted to the skin and subcutaneous structures to be treated. For example, it is even possible to produce ring-shaped pressure patterns of very strong cooling, so that the cells within the ring-shaped pressure pattern die but the cells in the center do not. Such structures recur in various skin lesions. With conventional devices working on the “all or nothing” (on-off) principle, it was not possible to treat such structures in the gentlest possible way, because all the tissue on which the cryogenic fluid was applied died, although often only a partial area really needed to be removed. In contrast, the use of a print head with several individually controllable nozzles makes it possible to treat even complex skin lesions, including those that are approximately ring-shaped and enclose healthy tissue in the center, in such a way that the healthy cells in the center are cooled to a lesser extent than the cells of the ring-shaped surrounding diseased tissue. Thus, a targeted treatment that is gentler for the patient is possible.

The heat loss from human skin in contact with air is insignificant, as air is an excellent thermal insulator. Application of the cryogenic liquid causes severe cooling of the surface to which the droplets are applied, with the cold also spreading to the underlying tissue, especially if cryogenic liquid droplets continue to be applied to this surface location by the print head.

Lateral thermal diffusion and cooling by blood perfusion favored the spread of cooling to the interior of the tissue, although this may take a few seconds. The cryogenic agent applied to the skin creates a heat sink below the surface of the skin, which is formed as a temperature gradient. The steeper the gradient, the faster a given amount of heat is extracted. Consequently, in order to achieve success, the cryomedium should produce a large drop in surface temperature as quickly as possible.

FIG. 9 shows a manually operable device 800 for applying the cryogenic liquid in vitro. For example, two-dimensional 2D or 3D cell clusters, e.g., bacterial cell clusters (“biofilm”), artificial skin, artificial cartilage, clusters of liver cells, muscle cells, and the like, can be increasingly grown in vitro, in this case, for example, in a Petri dish 802. By manual and preferably repeated application of a cryogenic fluid to specific locations on the cell cluster, the growth of the cells, including three-dimensional growth, can be controlled. In this regard, the printhead 706 is manually positioned on the areas of the cell assembly that are to be destroyed or slowed in growth. Cell culture dishes of various sizes and formats can be used instead of a Petri dish. In addition to medical and cosmetic purposes, a highly localized application of a cryogenic liquid can also be used for scientific questions, e.g. to slow down the metabolism and growth of cells in specific areas or to selectively preserve cell metabolism in a selected region by very rapid cooling to below 0° C. for further analyses.

FIG. 10 shows a device 900 for automatically applying the cryogenic liquid via a printhead 706 in vitro. The device 900 is rigidly or movably (e.g., rotatably and/or pivotally) attached to a unit for moving the device 902. The unit for moving the device 902 may be, for example, a robotic arm or other automatically operated unit for mechanically moving the device 900 within a plane x, y that is substantially parallel to a plane spanned by the underside of the cell culture vessel, and optionally additionally for mechanically moving the device 900 along a dimension y that is substantially orthogonal to the plane x, y. The combination of automatic positioning of the device with automatic droplet application of the cryogenic liquid via the printhead 706 may have the advantage of increasing control over exactly where a cryogenic liquid is applied. Thus, corresponding embodiments are particularly suitable for high precision applications. Preferably, the printhead 706 has a plurality of individually controllable print nozzles.

In one embodiment, the device and the unit for moving the device 902 are an integral part of an incubator or other device used for temperature control and, optionally, agitation of one or more prokaryotic or eukaryotic cell cultures. This may have the advantage of allowing the device to be periodically positioned on specific surface areas of the cell assembly for application of the cryogenic fluid and control of cell growth. Since both device 900 and unit for moving the device 902 are part of the incubator or apparatus, cryogenic treatment can be performed repeatedly without increasing the risk of infection.

### List of Reference Signs

100 Device
102 Control unit
103 Cryogenic liquid
104 Printer unit
105 Apparatus for manufacturing a component
106 Printhead
107 Motion unit
108 Container
109 Structuring unit
110 fluidic line
111 Droplet of cryogenic liquid
112 Surface
113 Object
114 Hardware component
116 Input device
118 Input device
120 Screen
122 graphical user interface
124 Control elements
126 Distribution scheme
127 Surface image
700 Device for medical or cosmetic cryotreatment
702 Container with cryogenic liquid
704 Control unit
706 Printhead
708 Droplets of cryogenic liquid
710 Skin
712 Surface
800 Device for in-vitro cryogenic treatment
802 Petri dish/cell culture vessel
900 Cryogenic treatment device
902 Unit for moving the device

1. Use of a cryogenic liquid, wherein the cryogenic liquid is applied droplewise to a surface in a digitally controlled manner for influencing a temperature of the surface.

2. The use according to claim 1, wherein the digitally controlled application of the cryogenic liquid controls a position of the dropletwise application in 2D or 3D, a volume of the dropletwise application, and/or an angle of the dropletwise application relative to the surface, wherein the volume and/or the angle may be position-dependent.

3. Use according to claim 1, wherein the cryogenic liquid is stored in a container fluidly coupled to a digital printhead, wherein the digital printhead is digitally controlled for dropletwise application of the cryogenic liquid to the surface.

4. (canceled)

5. Use according to claim 1, wherein the surface before application of the cryogenic liquid has a temperature of above −50° C., of above −20° C., in particular of above 0° C., or room temperature or body temperature of an animal or a human.

6. Use according to claim 1, wherein the surface has a temperature of 100° C. or less prior to application of the cryogenic liquid.

7. Use according to claim 1, wherein the application of the cryogenic liquid takes place under a protective atmosphere, in particular under a protective atmosphere with increased or decreased oxygen content relative to the normal atmosphere.

8. Use according to claim 1, wherein the application of the cryogenic liquid is performed under positive pressure or negative pressure.

9. Use according to claim 1, wherein the cryogenic liquid is helium, hydrogen, nitrogen, argon, oxygen, carbon dioxide, or a mixture of one or more thereof.

10. The use according to claim 1, wherein the use further comprises:
Detecting a 2D and/or 3D structure of the surface and/or an object comprising the surface,
determining structural elements of the surface to which the cryogenic liquid is to be applied, depending on the detected 2D and/or 3D structure; and
Generating control data adapted to control the application of the cryogenic liquid to the particular structural elements.

11. The use according to claim 10, wherein the use further comprises creating a digital distribution scheme for applying the cryogenic liquid to the determined structural elements depending on the detected 2D and/or 3D structure, wherein the control data is adapted to control the application of the cryogenic liquid to the determined structural elements according to the distribution scheme.

12. (canceled)

13. Use according to claim 1, wherein the surface is living or dead biological material, wherein the biological material comprises in particular: microorganisms, a cell culture and/or a cell cluster, in particular an in vivo or in vitro cell cluster, in particular an in vitro cell cluster for the growth of an artificial organ or organ part, wherein the biological material is in particular human or animal skin or a tissue sample.

14. Use according to claim 1, wherein the surface is a human or animal skin in vivo or in vitro.

15. (canceled)

16. Use according to claim 1, wherein the surface is a substrate, wherein one or more structuring steps are carried out to structure the surface and/or to apply a two-or three-dimensional structure to the surface, wherein at least before one of the structuring steps the application of the cryogenic liquid is carried out to influence the subsequent structuring step, or wherein a structuring step is carried out by applying the cryogenic liquid.

17-21. (canceled)

22. Device configured to perform the use of a cryogenic liquid according to claim 1.

23. A device for applying a cryogenic liquid to a surface to influence a temperature of the surface, comprising a container for holding the cryogenic liquid a digital print head fluidly coupled to the container, and a control unit for controlling the print head to apply the cryogenic liquid dropletwise to the surface.

24. The device according to claim 15, wherein the device is a medical device for selectively applying the cryogenic liquid to an organ or an organ part, in particular an organ or organ part cultured in vivo or in vitro, in particular human or animal skin, or wherein the device is a device for selectively applying the cryogenic liquid to individual cells, in particular nerve cells.

25. The device of claim 15, wherein the device is configured as:
an apparatus movable in two or three dimensions for applying the cryogenic liquid to the individual cells and/or the organ or organ part, in particular an organ or organ part cultivated in vitro, the apparatus preferably comprising a temperature sensor and being designed to detect the surface temperature of the organ or organ part during the application using the temperature sensor, wherein the surface temperature is used to control the application; and/or
a tool having a shaping that enables guiding and positioning of the printhead with one hand, the shaping being in particular in the form of a pen, painting or drawing instrument; and/or
an endoscope; and/or
an endoscope having a camera, the camera being mounted at the same end of the device as the printhead and oriented such that the camera is adapted to capture images of the printhead and the surface contacted thereby.

26. The device of claim 15, wherein the printhead includes a plurality of individually controllable nozzles, each of the nozzles configured to control the size and/or frequency of droplets ejected from the respective nozzle in response to control data from the control device.

27. The device of claim 15, further comprising a temperature sensor configured to detect, preferably without contact, the surface temperature of the substrate onto which the cryogenic fluid is applied, wherein the apparatus is configured to detect the surface temperature of the organ or organ part during the application using the temperature sensor, wherein the surface temperature is used to control the application such that the detected surface temperature is within a desired temperature range.

28. A apparatus for manufacturing a component, in particular an electronic component, comprising a device according to claim 15, wherein the surface is a substrate, wherein the apparatus performs one or more structuring steps in the course of manufacturing the component, in order to structure the surface and/or to apply a two-or three-dimensional structure to the surface, wherein at least prior to one of the structuring steps the application of the cryogenic liquid by the device takes place in order to influence the subsequent structuring step, or wherein a structuring step takes place as a result of the application of the cryogenic liquid.
